(19) United States
(12) Patent Application Publication
(10) Pub. No.: US 2022/0113798 A1
(43) Pub. Date: Apr. 14, 2022

(54) APPARATUS HAVING A HANDLE AND INFLATION BLADDERS

(71) Applicant: Hewlett-Packard Development Company, L.P., Spring, TX (US)

(72) Inventors: Sarthak Ghosh, Palo Alto, CA (US); Eric Faggin, Palo Alto, CA (US); Mithra Vankipuram, Palo Alto, CA (US)

(73) Assignee: Hewlett-Packard Development Company, L.P., Spring, TX (US)

(21) Appl. No.: 17/414,996

(22) PCT Filed: Jun. 26, 2019
(86) PCT No.: PCT/US2019/039236

§ 371 Date: Jun. 17, 2021

(51) Int. Cl. G06F 3/01; A61B 5/0533; A61B 5/00; A61B 5/0205; A61B 5/024

(52) U.S. Cl. CPC G06F 3/015; A61B 5/0533; A61B 5/6843; A61B 2562/0247; A61B 5/0205; A61B 5/02427; A61B 5/02438; A61B 5/6897

(57) ABSTRACT

In some examples, an apparatus can include a handle having a first surface and a second surface, an inflatable bladder located on the second surface, and a sensor located on the apparatus, where inflation of the inflatable bladder causes the sensor to be in contact with an outer surface of a user of the apparatus.

### BACKGROUND

Extended reality (XR) devices may be used to provide an altered reality to a user. An XR device may include a virtual reality (VR) device, a mixed reality (MR) device, and/or an augmented reality (AR) device. XR devices may include displays to provide a “virtual and/or augmented” reality experience to the user by providing video, images, and/or other visual stimuli to the user via the displays. XR devices may include audio output devices to provide audible stimuli to the user to further the virtual reality experienced by the user. XR devices may include hand-held XR devices to supplement the extended reality experience by a user. For example, a hand-held XR device may be used to virtually animate hand motions by the user, such as movement, grasping, releasing, etc.

### BRIEF DESCRIPTION OF THE DRAWINGS

FIG. 1 is a side view of an example of an apparatus having a handle and inflation bladders consistent with the disclosure.

FIG. 2 is a side view of an example of a controller of an XR device having a handle and inflation bladders consistent with the disclosure.

FIG. 3A is a side view of an example of a controller of an XR device having a handle and inflation bladders in a deflated state consistent with the disclosure.

FIG. 3B is a side view of an example of a controller of an XR device having a handle and inflation bladders in an inflated state consistent with the disclosure.

FIG. 4 is a side view of an example system and a controller consistent with the disclosure.

### DETAILED DESCRIPTION

XR devices may provide an altered reality to a user by providing video, audio, images, and/or other stimuli to a user via a display. As used herein, the term “XR device” refers to a device that provides a virtual, mixed, and/or augmented reality experience for a user.

The XR device may be experienced by a user through the use of a head mount device (e.g., a headset) and/or a hand-held XR device. For example, a user may wear the headset in order to view the display of the XR device and/or experience audio stimuli of the XR device, and/or utilize the hand-held XR device to virtually animate hand motions by the user, such as movement, grasping, releasing, etc. As used herein, the term “extended reality” refers to a computing device generated scenario that simulates experience through senses and perception. In some examples, an XR device may cover a user's eyes and provide visual stimuli to the user via a display, thereby substituting an “extended” reality (e.g., a “virtual reality”, a “mixed reality”, and/or an “augmented reality”) for actual reality. In some examples, an XR device may cover a user's ears and provide audible stimuli to the user via audio output devices to enhance the virtual reality experienced by the user. In some examples, an XR device may provide an overlay transparent or semi-transparent screen in front of a user's eyes such that reality is “augmented” with additional information such as graphical representations and/or supplemental data. For example, an XR device may overlay transparent or semi-transparent weather information, directions, and/or other information on an XR display for a user to examine.

A hand-held XR device may be used in conjunction with the XR device and can be a useful way to animate hand motions by a user. As used herein, the term “animation” refers to a dynamic visual medium produced from sequenced images that are manipulated to appear as motion. For example, while experiencing an extended reality via a headset, a user can utilize a hand-held XR device to simulate hand motions, which can be animated and presented for the user via the screen(s) included with the XR headset.

Monitoring physiological data of a user while the user is in an extended reality experience can yield information which can help developers understand a user's emotions and/or cognitive load while the user is in the extended reality experience. Utilizing sensors on a hand-held XR device can allow for this physiological information to be obtained. For example, a sensor in contact with an inner palm and/or on a fingertip of a user can yield physiological data which may be useful for XR developers. For instance, generating, modifying, and/or adjusting an animation may be done based on a physiological state of a user. For example, the controller 314 may generate, speed up, and/or slow down animations based on the physiological state of the user, among other examples.

In order to obtain useful data, sensors may have to achieve sufficient contact with a user. For example, a sensor may have to achieve a certain level of skin contact in order to obtain accurate physiological data from the user. However, in some instances a user may be using the hand-held XR device in such a manner (e.g., aggressively, vigorously, lazily, etc.) as to cause insufficient skin contact between the user and the sensor. Insufficient contact between the user and the sensor may yield inaccurate or no physiological data from the user.

An apparatus having a handle and inflation bladders, according to the disclosure, can allow for a hand-held XR device to ensure sufficient contact between a user and a sensor included on the hand-held XR device to yield physiological data from the user. Utilizing an inflation bladder can cause the sensor to make sufficient contact with the skin of the user so that no matter the manner of use by the user of the hand-held XR device (e.g., aggressively, vigorously, lazily, etc.), the sensor can maintain sufficient contact with the skin of the user so that accurate physiological data describing the user's extended reality experience can be obtained.

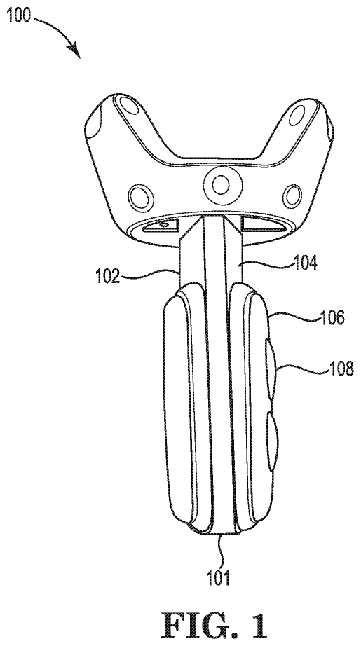

FIG. 1 is a side view of an example of an apparatus 100 having a handle 101 and inflation bladders 106 consistent with the disclosure. Apparatus 100 can include a handle 101, a first surface 102 of the handle 101, a second surface 104 of the handle 101, an inflatable bladder 106, and a sensor 108.

As illustrated in FIG. 1, apparatus 100 can include a handle 101. As used herein, the term “handle” refers to a member which can be grasped or held by a hand of a user. For example, a user may interact with apparatus 100 by grasping and/or holding the handle 101 with their hand.

The handle 101 can include a first surface 102 and a second surface 104. When a user holds the handle 101, at least one of the user's finger tips can be oriented substantially adjacent to the first surface 102 and the user's palm can be oriented substantially adjacent to the second surface 104.

The handle 101 can include an inflatable bladder 106 located on the second surface 104. As used herein, the term “bladder” refers to an inflatable container. For example, inflatable bladder 106 can be inflated with a fluid such that the inflatable bladder 106 includes fluid within the inflatable bladder 106. A fluid can include, for example, a gas, liquid (e.g., water or other liquids), etc.

In some examples, the inflatable bladder 106 can be filled with air. However, examples of the disclosure are not so limited. For example, the inflatable bladder 106 can be inflated with any other gas.

The inflatable bladder 106 can be located on the second surface 104 such that the user's palm can be oriented substantially adjacent to the inflatable bladder 106 when the user holds the handle 101. Orienting the inflatable bladder 106 so that the inflatable bladder 106 is oriented adjacent to the user's palm can allow for a sensor 108 to be in sufficient contact with the user, as is further described herein.

The apparatus 100 can include a sensor 108. As used herein, the term “sensor” refers to a device to detect events and/or changes in its environment and transmit the detected events and/or changes for processing and/or analysis. For example, the sensor 108 can detect events/changes related to the user holding the apparatus 100. The sensor 108 can be a biometric sensor, a touch sensor, and/or a pressure sensor, as is further described herein. The sensor 108 can be used to measure a physiological state of the user. As used herein, the term “physiological state” refers to a condition of a body of a user. For example, the physiological state of the user can be a biometric signal measured by a biometric sensor that can communicate biometric information (e.g., physiological data) about the user interacting with the apparatus 100, as is further described herein.

The sensor 108 can be a biometric sensor located on the inflatable bladder 106. As used herein, the term “biometric sensor” refers to a device to detect events and/or changes related to a person based on a physiological and/or behavioral characteristic. For example, the biometric sensor can detect events/changes related to a person based on a physiological and/or behavior characteristic. For example, the biometric sensor 106 can detect events/changes related to the user holding the apparatus 100, as is further described herein.

The sensor 108 can be a touch sensor located on the inflatable bladder 106. As used herein, the term “touch sensor” refers to a device to detect events and/or changes related to a person based on capacitive sensing by detecting an interaction by an object that is conductive and/or has a dielectric different from air. The touch sensor can, in some examples, be a metal electrode. In some examples, the metal electrode may be included as part of (e.g., on) the biometric sensor. In some examples, the touch sensor (e.g., the metal electrode) may be a standalone sensor. The touch sensor can detect when a user is holding the apparatus 100 based on a user's skin contacting the touch sensor.

The sensor 108 can be a pressure sensor included in the inflatable bladder 106. As used herein, the term “pressure sensor” refers to a device to detect events and/or changes related to a person by detecting a force applied to the inflatable bladder 106. For example, a user holding the apparatus 100 may apply a force to the inflatable bladder 106 (e.g., via the user's fingers and/or hand), and the pressure sensor can detect the force applied by the user.

As described above, in some instances, a user may be using the apparatus 100 in a manner (e.g., aggressively, vigorously, lazily, etc.) as to cause insufficient skin contact between the user and the sensor 108. For example, a user may be playing a game while in the extended reality experience and may be vigorously using the apparatus 100. During such motions, the user's grip on the apparatus 100 may change which may cause insufficient skin contact between the sensor 108 and the user.

Accordingly, inflation of the inflatable bladder 106 can cause the sensor 108 to be in contact with an outer surface of the user of the apparatus 100. For example, in an instance in which the user's grip changes causing insufficient skin contact between the sensor 108 and the user, inflation of the inflatable bladder 106 can cause the sensor 108 to move to close a space between the user's hand and the sensor 108. Such movement can position the sensor 108 adjacent to the user's hand so that sufficient skin contact between the sensor 108 and the user's hand (e.g., the user's palm) can be achieved. In other words, as inflatable bladder 106 is inflated, sensor 108 can be caused to be in contact with the surface of the user. For example, the sensor 108 can come into contact with the user's skin as a result of inflatable bladder 106 being in an inflated state. As used herein, the term “inflated state” refers to a state in which a container (e.g., an inflatable bladder) includes gas and/or fluid provided by an inflation mechanism.

Inflatable bladder 106 can be inflated via an inflation mechanism. As used herein, the term “inflation mechanism” refers to a device which causes a bladder to expand and/or distend with gas and/or fluid. For example, the inflation mechanism can inflate inflatable bladder 106 by forcing gas and/or fluid into inflatable bladder 106. The inflation mechanism can include a bellows, mechanical lever, electric motor powering a pump and an accompanying valve system, among other types of inflation mechanisms.

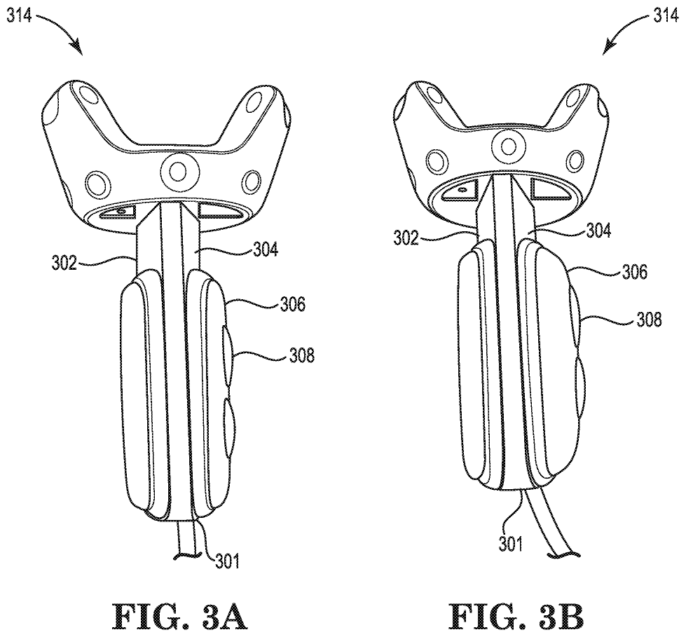

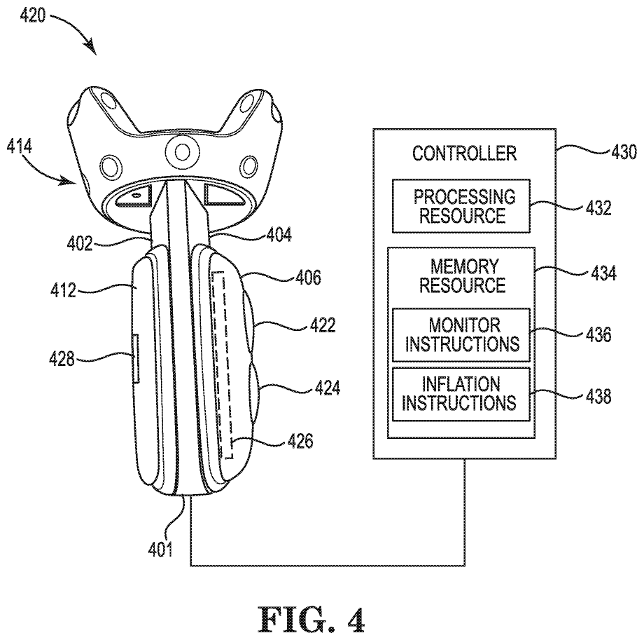

The inflatable bladder 106 can be inflated in response to a sensor measurement of the sensor 108 being less than a threshold amount. For example, the sensor 108 can be a biometric sensor, touch sensor, and/or pressure sensor, and can cause the inflatable bladder 106 to be inflated in response to a biometric sensor measurement, touch measurement, and/or pressure measurement, respectively, being less than a threshold amount, as is further described in connection with FIG. 3A, FIG. 3B, and FIG. 4.

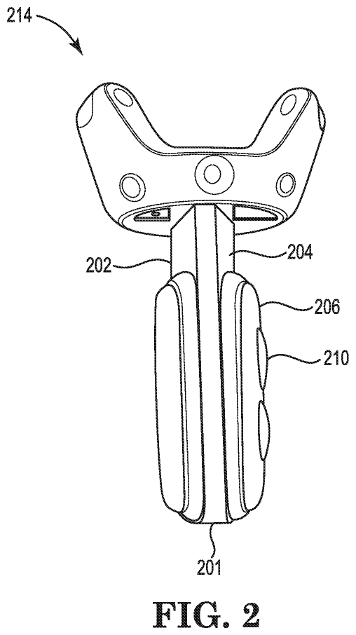

FIG. 2 is a side view of an example of a controller 214 of an XR device having a handle 201 and inflation bladders 206 consistent with the disclosure. The controller 214 of the XR device can include a handle 201, a first surface 202 of the handle 201, a second surface 204 of the handle 201, an inflatable bladder 206, and a biometric sensor 210.

As previously described in connection with FIG. 1, the controller 214 of the XR device can include a handle 201 having a first surface 202 and a second surface 204. The second surface 204 can be located opposite the first surface 202.

The second surface 204 can include the inflatable bladder 206. For example, the inflatable bladder 206 can be located on the second surface 204 of the handle 201.

The inflatable bladder 206 can include a biometric sensor 210. As used herein, the term “biometric sensor” refers to a device to detect events and/or changes related to a person based on a physiological and/or behavioral characteristic. For example, the biometric sensor can detect events/changes related to the user holding the controller 214 of the XR device.

In some examples, the biometric sensor 210 can be a galvanic skin response (GSR) sensor. As used herein, the term “galvanic skin response sensor” refers to a sensor which measures variations in electrical characteristics (e.g., electrodermal activity) of a user's skin. For instance, the GSR sensor can measure a user's skin for conductance. The GSR sensor can determine an intensity of a user's emotional state. Galvanic skin response, as a signal, can be used to determine states of relaxation in the user or mental workload a user may be experiencing while in virtual reality, among other examples.

In some examples, the GSR sensor can cause the inflatable bladder 206 to be inflated. For example, in response to a GSR measured by the GSR sensor being less than a threshold GSR amount, a controller can cause the inflatable bladder 206 to be inflated, as is further described in connection with FIG. 3A, FIG. 3B, and FIG. 4.

Inflation of the inflatable bladder 206 can cause the biometric sensor 210 to be in contact with an outer surface of a user of the controller 214 of the XR device. For example, in some instances a user may be using the controller 214 of the XR device in a manner (e.g., aggressively, vigorously, lazily, etc.) as to cause insufficient skin contact between the user and the biometric sensor 210. Inflation of the inflatable bladder 206 can cause the biometric sensor 210 to be in contact with the user (e.g., the user's palm) while the inflatable bladder 206 is in an inflated state. This can allow the biometric sensor 210 to make sufficient contact with the user so as to be able to yield physiological data from the user no matter the manner of use by the user of the controller 214 of the XR device.

FIG. 3A is a side view of an example of controller 314 of an XR device having a handle 301 and inflation bladders 306 in a deflated state consistent with the disclosure. The controller 314 of the XR device can include a handle 301, a first surface 302 of the handle 301, a second surface 304 of the handle 301, an inflatable bladder 306, and a sensor 308.

As illustrated in FIG. 3A, the inflatable bladder 306 is in a deflated state. As used herein, the term “deflated state” refers to a state in which a container (e.g., an inflatable bladder) does not include gas and/or fluid provided by an inflation mechanism. In the deflated state illustrated in FIG. 3A, in some examples the sensor 308 may not make sufficient contact with a user. As a result, the sensor 308 may not be able to obtain physiological data about a user holding the controller 314 of the XR device. Accordingly, the inflatable bladder 306 may be inflated, as is further described herein.

In some examples, the sensor 308 can be a GSR sensor. The GSR sensor can measure a user's skin for conductance in order to determine an intensity of a user's emotional state. In response to the GSR measured by the GSR sensor being less than a threshold GSR amount, a controller (e.g., as is further described in connection with FIG. 4) can cause the inflatable bladder 306 to inflate. For example, in response to the user's skin conductance being less than a threshold conductance amount, the controller can cause the inflatable bladder 306 to inflate from the deflated state (e.g., as illustrated in FIG. 3A) to an inflated state (e.g., as is further described in connection with FIG. 3B).

In some examples, the sensor 308 can be a pressure sensor. The pressure sensor can measure a force applied by a user when the user is holding the controller 314 of the XR device. In response to the force measured by the pressure sensor being less than a threshold pressure, a controller (e.g., as is further described in connection with FIG. 4) can cause the inflatable bladder 306 to inflate from the deflated state (e.g., as illustrated in FIG. 3A) to an inflated state (e.g., as is further described in connection with FIG. 3B).

In some examples, the sensor 308 can be a touch sensor. The touch sensor can measure a capacitance caused by a user when the user is holding the controller 314 of the XR device. In response to the capacitance measured by the touch sensor being less than a threshold capacitance, a controller (e.g., as is further described in connection with FIG. 4) can cause the inflatable bladder 306 to inflate from the deflated state (e.g., as illustrated in FIG. 3A) to an inflated state (e.g., as is further described in connection with FIG. 3B).

FIG. 3B is a side view of an example of a controller 314 of the XR device having a handle 301 and inflation bladders 306 in an inflated state consistent with the disclosure. The controller 314 of the XR device can include a handle 301, a first surface 302 of the handle 301, a second surface 304 of the handle 301, an inflatable bladder 306, and a sensor 308.

As illustrated in FIG. 3B, the inflatable bladder 306 is in an inflated state. In the inflated state illustrated in FIG. 3B, in some examples the sensor 308 can make sufficient contact with a user. As a result, the sensor 308 can obtain physiological data about the user holding the controller 314 of the XR device.

As previously described in connection with FIG. 3A, a controller can cause the inflatable bladder 306 to inflate in response to the GSR sensor, the pressure sensor, and/or the touch sensor sensing a threshold condition being met. In some examples, in response to the user's skin conductance (e.g., measured by a GSR sensor) being less than a threshold conductance amount, the controller can cause the inflatable bladder 306 to inflate. In some examples, in response to the force measured by the pressure sensor being less than a threshold pressure, the controller can cause the inflatable bladder 306 to inflate. In some examples, in response to the capacitance measured by the touch sensor being less than a threshold capacitance, the controller can cause the inflatable bladder 306 to inflate.

Although the controller 314 of the XR device is described above as including an inflatable bladder 306 located on the second surface 304, examples of the disclosure are not so limited. For example, the controller 314 of the XR device can include an inflatable bladder located on the first surface 302 which can inflate to cause the sensor 308 to contact the outer surface of the user of the controller 314 of the XR device, as is further described in connection with FIG. 4.

As illustrated in FIG. 3B, the inflatable bladder 306 can be inflated to a maximum inflation amount of the inflatable bladder 306. The maximum inflation amount of the inflatable bladder 306 can be, for example, a maximum threshold inflation beyond which damage would occur to the inflatable bladder 306 as a result of over-inflation. However, in some examples, inflation of inflatable bladder 306 can be limited prior to the inflatable bladder 306 reaching the maximum inflation amount. In some examples the inflation of inflatable bladder 306 can be limited to an amount that is less than the maximum amount of inflation of inflatable bladder 306.

In some examples, the controller 314 of the XR device can include a pressure sensor (e.g., not illustrated in FIG. 3B) to sense a pressure on the inflatable bladder 306. In some instances, the pressure on the inflatable bladder 306 can be sensed by detecting the force applied by the user's fingers and/or hand when the user is holding the controller 314. In some instances, the pressure applied to the pressure sensor can be as a result of inflation of the inflatable bladder 306 itself (e.g., in combination with a force applied by the user's fingers and/or hand when the user is holding the controller 314, or without the force applied by the user's fingers and/or hand when the user is not holding the controller 314) and can be sensed using the pressure sensor included on the XR device as described above. In response to detecting a particular amount of pressure applied to the pressure sensor, a controller (e.g., controller 430, further described in connection with FIG. 4) can limit inflation of the inflatable bladder 306 to a predetermined amount of inflation that corresponds to the detected amount of pressure or deflate the inflatable bladder 306 if the particular amount of pressure is exceeded.

In some examples, the controller 314 of the XR device can utilize a touch sensor (e.g., as previously described in connection with FIGS. 1 and 3A, as well as further described in connection with FIG. 4) to determine whether a biometric signal of a user (e.g., detected by a biometric sensor of the controller 314 of the XR device previously described in connection with FIGS. 1, 2, and 3A, as well as further described in connection with FIG. 4) is at a threshold signal level. In response to detecting a biometric signal of the user that exceeds a threshold signal level, a controller (e.g., controller 430, further described in connection with FIG. 4) can limit inflation of the inflatable bladder 306 to a predetermined amount of inflation that corresponds to the detected amount of pressure.

In some examples, the inflatable bladder 306 can deflate. For example, the inflatable bladder 306 can be deflated via a deflation mechanism. As used herein, the term “deflation mechanism” refers to a device which causes a bladder to compress and/or contract by removing gas and/or fluid located in the bladder. For example, the deflation mechanism can deflate inflatable bladder 306 by removing gas and/or fluid from the inflatable bladder 306. The deflation mechanism can include a vacuum pump, external forces (e.g., letting the inflatable bladder 306 deflate on its own by contraction of a surface of the inflatable bladder 306, internal pressure of the inflatable bladder 306, etc.), among other deflation mechanisms.

The inflatable bladder 306 can be deflated in response to a condition being met. For example, the condition may be a touch sensor of the controller 314 of the XR device not detecting a user touching the touch sensor (e.g., the user is not holding the controller 314 of the XR device), among other examples.

FIG. 4 is a side view of an example system 420 and a controller 430 consistent with the disclosure. The system 420 can include handheld XR controller 414 and controller 430. The handheld XR controller 414 can include a handle 401, a first surface 402 of the handle 401, a second surface 404 of the handle 401, an inflatable bladder 406, 412, a GSR sensor 422, a touch sensor 424, a pressure sensor 426, and a photoplethysmography (PPG) sensor 428.

As previously described in connection with FIGS. 1 and 2, the handheld XR controller 414 can include a handle 401 having a first surface 402 and a second surface 404. The second surface 404 can be located opposite the first surface 402.

The second surface 404 can include the inflatable bladder 406. For example, the inflatable bladder 406 can be located on the second surface 404 of the handle 401.

In some examples, the inflatable bladder 406 is one of a set of inflatable bladders. For example, the first surface 402 can include the inflatable bladder 412. The inflatable bladder 412 can be located on the first surface 404 of the handle 401.

The inflatable bladder 406 can include a GSR sensor 422. The GSR sensor 422 can measure a user's skin for conductance and can determine an intensity of a user's emotional state. The GSR sensor 422 can be used to determine states of relaxation in the user or mental workload a user may be experiencing while in virtual reality, among other examples.

The inflatable bladder 406 can include a touch sensor 424. The touch sensor 424 can detect when a user is holding the handheld XR controller 414 based on a user's skin contacting the touch sensor 424.

The inflatable bladder can include a pressure sensor 426 included in the inflatable bladder 406. The pressure sensor 426 can detect a person holding the handheld XR controller 414 by detecting a force applied to the inflatable bladder 406. For example, a user holding the handheld XR controller 414 may apply a force to the inflatable bladder 406 (e.g., via the user's fingers and/or hand), and the pressure sensor 426 can detect the force applied by the user.

Although the inflatable bladders of the hand-held XR controller 414 are illustrated in FIG. 2 as including a GSR sensor 422, a touch sensor 424, and a pressure sensor 426, examples of the disclosure are not so limited. For example, the hand-held XR controller 414 can include any subset of the sensors 422, 424, and/or 426 (e.g., the hand-held XR controller 414 can include the GSR sensor 422 and the touch sensor 424, the GSR sensor 422 and the pressure sensor 426, the touch sensor 424 and the pressure sensor 426, just the GSR sensor 422, just the touch sensor 424, or just the pressure sensor 426, and/or any other combinations thereof).

In some examples, the handheld XR controller 414 can be connected to a controller 430. As described herein, the controller 430 may perform functions related to an apparatus having a handle and inflation bladders. Although not illustrated in FIG. 4, the controller 430 may include a processor and a machine-readable storage medium. Although the following descriptions refer to a single processor and a single machine-readable storage medium, the descriptions may also apply to a system with multiple processors and multiple machine-readable storage mediums. In such examples, the controller 430 may be distributed across multiple machine-readable storage mediums and the controller 430 may be distributed across multiple processors. Put another way, the instructions executed by the controller 430 may be stored across multiple machine-readable storage mediums and executed across multiple processors, such as in a distributed or virtual computing environment.

The processing resource 432 may be a central processing unit (CPU), a semiconductor based microprocessor, and/or other hardware devices suitable for retrieval and execution of machine-readable instructions 436, 438 stored in a memory resource 434. Processing resource 432 may fetch, decode, and execute instructions 436, 438. As an alternative or in addition to retrieving and executing instructions 436, 438, processing resource 432 may include a plurality of electronic circuits that include electronic components for performing the functionality of instructions 436, 438.

Memory resource 434 may be any electronic, magnetic, optical, or other physical storage device that stores executable instructions 436, 438 and/or data. Thus, memory resource 434 may be, for example, Random Access Memory (RAM), an Electrically-Erasable Programmable Read-Only Memory (EEPROM), a storage drive, an optical disc, and the like. Memory resource 434 may be disposed within controller 430, as shown in FIG. 4. Additionally and/or alternatively, memory resource 432 may be a portable, external or remote storage medium, for example, that causes controller 430 to download the instructions 436, 438 from the portable/external/remote storage medium.

The controller 430 may include instructions 436 stored in the memory resource 434 and executable by the processing resource 432 to monitor at least one of a pressure, a contact amount, and a GSR via the GSR sensor 422, the touch sensor 424, and the pressure sensor 426. In some examples, the controller 430 can monitor the GSR sensor 422 for conductance of a user's skin who is utilizing the handheld XR controller 414, measured by the GSR sensor 422. In some examples, the controller 430 can monitor the touch sensor 424 for a capacitance change caused by a user holding the handheld XR controller 414. In some examples, the controller 430 can monitor the pressure sensor 426 for a force applied by a user caused by a user holding the handheld XR controller 414.

The controller 430 may include instructions 438 stored in the memory resource 434 and executable by the processing resource 432 to cause inflation of the inflatable bladders 406 and/or 412 in response to a threshold condition being met, as is further described herein.

In some examples, the controller 430 can cause inflation of the inflatable bladders 406 and/or 412 in response to the GSR measured by the GSR sensor 422 being less than a threshold GSR amount. For example, in response to the user's skin conductance being less than a threshold conductance amount, the controller 430 can cause the inflatable bladder 406 and/or 412 to inflate from a deflated state to an inflated state.

In some examples, the controller 430 can cause inflation of the inflatable bladders 406 and/or 412 in response to a pressure measured by the pressure sensor 426 being less than a threshold pressure. For example, in response to the pressure measured by the pressure sensor 426 being less than a threshold pressure, the controller 430 can cause the inflatable bladder 406 and/or 412 to inflate from a deflated state to an inflated state.

In some examples, the controller 430 can cause inflation of the inflatable bladders 406 and/or 412 in response to the capacitance measured by the touch sensor 424 being less than a threshold capacitance amount. For example, in response to the capacitance measured by the touch sensor 424 being less than a threshold capacitance, the controller 430 can cause the inflatable bladder 406 and/or 412 to inflate from a deflated state to an inflated state.

In some examples, inflation of the inflatable bladder 412 can cause the GSR sensor 422 and/or the touch sensor 424 to contact the outer surface of the user of the handheld XR controller 414. Such contact can allow the GSR sensor 422 and/or the touch sensor 424 to yield accurate physiological data from the user describing the extended reality experience. Inflation of the inflatable bladder 412 can be done separately from inflation of the inflatable bladder 406 (e.g., the inflatable bladder 412 is inflated to an inflated state whereas the inflatable bladder 406 is left in a deflated state) or in conjunction with the inflatable bladder 406 (e.g., both the inflatable bladders 406 and 412 are inflated to an inflated state). In some examples, the handheld XR controller 414 can include the inflatable bladder 412 but not the inflatable bladder 406 (e.g., the handheld XR controller 414 does not include the inflatable bladder 406 and the GSR sensor 422 and/or the touch sensor 424 are located directly on the second surface 404), and the inflatable bladder 406 can be inflated to cause the GSR sensor 422 and/or the touch sensor 424 to contact the outer surface of the user of the handheld XR controller 414 to yield accurate physiological data from the user describing the extended reality experience.

As described above, the inflatable bladder 412 can include a photoplethysmography (PPG) sensor 428. As used herein, the term “photoplethysmography sensor” refers to a sensor which measures blood volume changes in a bed of tissue. For example, a PPG sensor can detect blood volume changes in a microvascular bed of tissue.

The controller 430 may further include instructions stored in the memory resource 434 and executable by the processing resource 432 to monitor changes in blood volume of a user's fingertip via the PPG sensor 428. For example, when a user holds the handheld XR controller 414, the user can place their fingertip on the PPG sensor 428. The PPG sensor 428 can, accordingly, monitor changes in blood volume of the user while the user is holding the handheld XR controller 414.

The controller 430 may further include instructions stored in the memory resource 434 and executable by the processing resource 432 to monitor a heart rate of the user of the handheld XR controller 414 via the PPG sensor 428. For example, by monitoring changes in the blood volume of the user, the controller 430 can determine a heart rate of the user of the handheld XR controller 414. Heart rate, as a signal, can be used to determine a user's emotional response to extended reality content. Additionally, it can also be used to determine states of relaxation in the user or mental workload a user may be experiencing while in extended reality, among other examples.

An apparatus having a handle and inflation bladders, according to the disclosure, can allow an inflation bladder to position sensors in such a manner so as to ensure sufficient contact between a user and the sensor. Ensuring sufficient contact between the user and the sensor can allow for accurate physiological data describing a user's extended reality experience to be obtained, no matter the manner of use by the user of the hand-held XR device (e.g., aggressively, vigorously, lazily, etc.).

In the foregoing detailed description of the disclosure, reference is made to the accompanying drawings that form a part hereof, and in which is shown by way of illustration how examples of the disclosure may be practiced. These examples are described in sufficient detail to enable those of ordinary skill in the art to practice the examples of this disclosure, and it is to be understood that other examples may be utilized and that process, electrical, and/or structural changes may be made without departing from the scope of the disclosure. Further, as used herein, “a” can refer to one such thing or more than one such thing.

The figures herein follow a numbering convention in which the first digit corresponds to the drawing figure number and the remaining digits identify an element or component in the drawing. For example, reference numeral 102 may refer to element 102 in FIG. 1 and an analogous element may be identified by reference numeral 202 in FIG. 2. Elements shown in the various figures herein can be added, exchanged, and/or eliminated to provide additional examples of the disclosure. In addition, the proportion and the relative scale of the elements provided in the figures are intended to illustrate the examples of the disclosure and should not be taken in a limiting sense.

It can be understood that when an element is referred to as being “on,” “connected to”, “coupled to”, or “coupled with” another element, it can be directly on, connected, or coupled with the other element or intervening elements may be present. In contrast, when an object is “directly coupled to” or “directly coupled with” another element it is understood that are no intervening elements (adhesives, screws, other elements) etc.

The above specification, examples and data provide a description of the method and applications, and use of the system and method of the disclosure. Since many examples can be made without departing from the scope of the system and method of the disclosure, this specification merely sets forth some of the many possible example configurations and implementations.

What is claimed is:

1. An apparatus, comprising:
a handle having a first surface and a second surface;
an inflatable bladder located on the second surface; and
a sensor located on the apparatus;
wherein inflation of the inflatable bladder causes the sensor to be in contact with an outer surface of a user of the apparatus.

2. The apparatus of claim 1, wherein the sensor is a biometric sensor located on the inflatable bladder.

3. The apparatus of claim 1, wherein the sensor is a touch sensor located on the inflatable bladder.

4. The apparatus of claim 1, wherein the sensor is a pressure sensor included in the inflatable bladder.

5. The apparatus of claim 1, wherein the inflatable bladder is inflated in response to a sensor measurement of the sensor being less than a threshold amount.

6. The apparatus of claim 1, wherein the inflatable bladder is inflatable via an inflation mechanism.

7. A controller of an extended reality (XR) device, comprising:
a handle having a first surface and a second surface, wherein the second surface is located opposite the first surface;
an inflatable bladder located on the second surface; and
a biometric sensor located on the inflatable bladder;
wherein inflation of the inflatable bladder causes the biometric sensor to be in contact with an outer surface of a user of the controller.

8. The controller of claim 7, wherein the controller further includes a pressure sensor such that the inflatable bladder is inflated in response to a pressure sensed by the pressure sensor being less than a threshold pressure amount.

9. The controller of claim 7, wherein the controller further includes a touch sensor such that the inflatable bladder is inflated in response to a contact amount sensed by the touch sensor being less than a threshold contact amount.

10. The controller of claim 7, wherein the biometric sensor is a galvanic skin response (GSR) sensor such that the inflatable bladder is inflated in response to a GSR sensed by the GSR sensor being less than a threshold GSR amount.

11. An extended reality (XR) device, comprising:
a handheld XR controller comprising:
a handle having a first surface and a second surface, wherein the second surface is located opposite the first surface;
an inflatable bladder located on the second surface;
a galvanic skin response (GSR) sensor located on the inflatable bladder;
a pressure sensor and a touch sensor; and
a controller including a memory resource and a processing resource to execute non-transitory machine-readable instructions stored in the memory resource to:
monitor at least one of a pressure, a contact amount, and a GSR via the GSR sensor, the pressure sensor, and the touch sensor, and
cause inflation of the plurality of inflatable bladders in response to a threshold condition being met.

12. The XR device of claim 11, wherein the processing resource executes the instructions to cause the inflation of the plurality of inflatable bladders in response to at least one of the GSR sensor, the pressure sensor, and the touch sensor sensing the threshold condition being met.

13. The XR device of claim 11, wherein the inflatable bladder is one of a set of inflatable bladders.

14. The XR device of claim 13, wherein a different one of the inflatable bladders of the set of inflatable bladders includes a photoplethysmography (PPG) sensor.

15. The XR device of claim 14, wherein the processing resource executes the instructions to monitor a heart rate of a user of the XR device via the PPG sensor.
